(19) United States
(12) Patent Application Publication
(10) Pub. No.: US 2006/0097422 A1
(43) Pub. Date: May 11, 2006

(54) Method for performing surgery and appliances produced thereby

(76) Inventor: Andrew Jay Diamond, Marietta, GA (US)

Correspondence Address: KLAUBER & JACKSON, 411 HACKENSACK AVENUE, HACKENSACK, NJ, 07601, US

(21) Appl. No.: 11/268,779

(22) Filed: Nov. 7, 2005

Related U.S. Application Data
(60) Provisional application No. 60/625,494, filed on Nov. 8, 2004.

(51) Int. Cl. A61B 5/107; A61F 2/02; B29C 33/42; A61F 5/08

(52) U.S. Cl. 264/222; 600/587; 623/23.72; 602/17

(57) ABSTRACT

The present invention provides a method for performing plastic surgery comprising the steps of: obtaining a substantially three dimensional image of the target body part or area; manipulating the substantially three dimensional image of the target body part or area into a desired shape or profile; and making a mold of the desired shape or profile. The method may further comprise using the mold of the desired shape or profile during the procedure as a guide for placing an implant into the target body part or area and/or using the mold of the desired shape or profile after the plastic surgery to effect or maintain the desired shape or profile of the target body part or area. The present invention further extends to a mold of the desired shape or profile made according to the procedure.

### CROSS REFERENCE TO RELATED APPLICATIONS

The present application is a utility application claiming priority to co-pending provisional application Ser. No. 60/625,494, filed Nov. 8, 2004. Applicant claims the benefits of 35 U.S.C. §119(e) as to the provisional application, and the disclosure of the application is incorporated herein in its entirety.

### FIELD OF THE INVENTION

The invention relates to a method for preparing surgical appliances using computerized modeling as well as to new surgical appliances prepared thereby. More particularly, this invention relates in general to plastic surgery, in both its preoperative planning and postoperative retention. This invention provides a method and system for three dimensional scanning of a preoperative patient for the purpose of presenting various morphings of a visage in order to create an intraoperative negative sizing mold that is also used postoperatively for retention.

### BACKGROUND OF THE INVENTION

Aesthetic Facial Surgery, ed. Charles J. Krause; J.B Lippincott Company, 1991 provides a review of many of the procedures and complexities of rhinoplasty. Many innovations for nasal implants and polyethylene devices are provided in the prior art. Although these innnovations are suitable for the purposes they address, they differ from the present invention as contrasted herein. A summary of innovations somewhat relevant to the instant invention at hand assists in distinguishing the novelty of the present methods and appliances.

U.S. Pat. No. 4,938,234 provides a method for surgically implanting a contour nasal implant into a nose of a patient to elevate the nasal tip and to augment and to improve the frontal and profile views of the patient's nose. The method comprises the steps of drawing, onto the nose of a patient into which a contour nasal implant is to be surgically implanted, a line connecting the highest points of the superior palpebral sulci such that the line crosses the nasal dorsum at a point representing the nasion and the highest point of implant insertion; placing two pledgets in each nostril in the nose of the patient; performing an open rhinoplasty incision into the skin located between the nostrils of the patient; retracting superiorly the columella skin off of the lower lateral cartilages on each side of the nostril up to the highest point of the natural dome of the nose of the patient; dissecting the skin off of the dorsum of the nose to the line previously drawn across the nasal dorsum; inserting a contour nasal implant having an elongated central member having a dorsal section, a predetermined shaped tip and a keel having a selected width and length which is located under the predetermined shaped tip with the keel thereof extending downward between the lower lateral cartilage until the desired implantation position is obtained; and suturing the rhinoplasty incision in the skin between the nostrils.

U.S. Pat. No. 5,030,232 provides a nasal implant characterized by a composition of hard-grade silicone fashioned into a special shape. One variation in the composition material is to use soft silicone only for the tip in order to overcome the fear of some surgeons to use hard silicone in this particular area. A special and improved shape of the implant, in the form of a modified hourglass, provides a well rounded and larger tip portion thanb that of the prior art. The supra-tip of this improved implant shows a depression dorsally, is less wide than the rest of the implant and has a pronounced slope laterally. These features, particularly at the supra-tip region avoid the unnatural effects of the prior art devices. Moreover this implant has a variable thickness form upper end to tip—that is it starts thin, increases in thickness and then goes thinner again—whereas the prior art starts thin and continually increases throughout.

U.S. Pat. No. 5,112,353 provides a contour nasal implant adapted to be used in rhinoplasty surgery. The contour nasal implant includes an elongated, central member having a first end and a second end wherein the second end is spaced a predetermined distance from the first end. The first end of the elongated central member is flared to define a dorsal support end or dorsal section which is adapted to be positioned over the nasal dorsum in the nose of a patient to augment the frontal and profile views of the nose along the nasal dorsum. The second end includes a tip having a predetermined shape which is located on the same side of the elongated central member as the dorsal support end. A second end includes a keel having a selected width and length and the keel is located on the second end under the predetermined shaped tip. The keel is positioned substantially perpendicular to the elongated central member and is positioned such that the length of the keel is generally parallel to the elongated central member. The tip is operative, when implanted into the nose of a patient, to reshape and elevate the nasal tip of the nose of a patient. The tip and the dorsal support end contour and shape the frontal and profile views of the nose of a patient.

U.S. Pat. No. 4,994,084 describes a homograft implant made from prepackaged, processed homograft material. The homograft material is taken in its purchased dehydrated form and cut into strips of varying shapes and sized. The strips are then adhesively secured together in a laminar manner by a tissue adhesive to form the desired shape and density of the implant. Once the implant has been made, it may then be surgically implanted in a desired location, immediately beneath the patient's dermis so as to alter the exterior appearance of the patient at the implant site. Since the implant is formed of dehydrated homograft material, endogenous tissue readily attaches to the implant after a short period of time, so that the implant becomes integral with the body.

U.S. Pat. No. 5,876,435 provides an improved coupling method and device for an ocular prosthesis to a porous polyethylene implant is provided. According to the invention, a small surgical screws having a domed head is inserted into a porous plastic implant after the implant has been implanted into an enucleated orbit allowed to vascularize. The domed head projects from the anterior surface of the implant which is covered by conjunctive tissue and is received by a complementary cavity on the rear surface of the ocular prosthesis.

U.S. Pat. No. 5,053,050 provides a defect in cartilage or bone, or both, to excize damaged or pathological tissue, and it is filled with an implant having capability for complete regeneration of the skeletal tissue as a chondrogenic or osteogenic phenotype. The implant comprises cells expressing a chondrocyte phenotype (80. times. 10. sup.6 cells/ml) embedded in a biocompatible matrix having about 20% serum, which provides a permissive environment for maturation and transformation of the implant to a fully integrated state with the surrounding tissue. A portion of the implant may comprise a bone segment or a bone substitute.

U.S. Pat. No. 5,786,217 provides methods and compositions for the repair of articular cartilage defects in a mammal. Denuded chondrogenic cells are proliferated ex vivo as monolayer cultures in order to expand the pool of available chondrogenic cells. During proliferation the chondrogenic cells stop secreting the extracellular matrix components, type II collagen and sulfated proteoglycans. The proliferated cells then are seeded into a pre-shaped well having a cell contacting, cell abhesive surface. The cells cultured in the well redifferentiate and begin to secrete cartilage-specific extracellular matrix again. Accordingly, essentially unlimited amounts of synthetic cartilage may be prepared from small samples of biopsy tissue. Also provided are methods for surgically repairing articular cartilage defects in mammals using the synthetic cartilage prepared in accordance with the invention.

U.S. Pat. No. 5,842,477 relates to methods of making and/or repairing cartilage in vivo comprising implanting into a patient, at a site of cartilage damage or loss, a biocompatible, non-living three-dimensional scaffold or framework structure in combination with periosteal/perichondrial tissue that can be used to hold the scaffold in place and provides a source of chondrocyte progenitor cells, chondrocytes and other stromal cells for attachment to the scaffold in vivo. In addition, a preparation of cells that can include chondrocytes, chondrocyte progenitor cells or other stromal cells is administered, either before, during or after implantation of the scaffold and/or the periosteal perichondrial tissue; the cells are administered directly into the site of the implant in vivo and promote the induction of factors that enhance chondrogenesis and the migration of chondrocytes, progenitor cells and other stromal cells from the adjacent in vivo environment into the scaffold for the production of new cartilage at the site of implantation.

U.S. Pat. No. 5,916,557 relates to the use of an ECM-altering enzymatic activity, such as a proteoglycanase or a protease, to stimulate the generation of cartilage tissue by inducing chondrocytes to synthesize new cartilage matrix. It has been discovered that treating chondrocytes with an enzymatic activity that modifies the territorial ECM of the cell, especially cell surface proteoglycans, can in and of itself be sufficient to stimulate cartilage production by the chondrocytes. The subject invention can be employed therapeutically to correct or prevent degeneration of connective tissue. For instance, the present method can be used in the treatment of disorders comprising cartilage such as found in a diarthroidal joint (e.g. articular and interarticular cartilage), as well as in the treatment of tendon and ligamental tissues. Such disorders can range from chronic degeneration brought about by disease, overuse, or trauma, to plastic or reconstructive surgery. Moreover, the subject method may also be applied to both the development and implantation of prosthetic devices.

U.S. Pat. No. 5,041,138 describes methods and artificial matrices for the growth and implantation of cartilaginous structures and surfaces. In the preferred embodiments, chondrocytes are grown on biodegradable, biocompatible fibrous polymeric matrices. Optionally, the cells are proliferated in vitro until an adequate cell volume and density has developed for the cells to survive and proliferate in vivo. One advantage of the matrices is that they can be cast or molded into a desired shape, on an individual basis, so that the final product closely resembles a patient's own ear or nose. Alternatively, flexible matrices can be used which can be manipulated at the time of implantation, as in a joint, followed by remodeling through cell growth and proliferation in vivo. The cultured cells can also be maintained on the matrix in a nutrient media for production of bioactive molecules such as angiogenesis inhibiting factor.

U.S. Pat. No. 6,001,352 provides a method for stimulating chondrocyte proliferation and inhibiting chondrocyte differentiation along the endochondral developmental pathway is provided comprising contacting condrocytes with an effective amount of Platelet-Derived Growth Factor (PDGF) such as PDGF-BB, PDGF-AA OR PDGF-AB in the substantial absence of growth factors which promote cell differentiation. This allows such cells to be multiplied in culture for loading onto a scaffolding material and implanting into a cartilage or bone wound.

As outlined above, the prior art patents that relate to implants and nasal reconstructive surgeries largely entail elements such as a nasal implant with composition of hard-grade silicone fashioned into a special shape with soft silicone used only for the tip; a homograft implant made from prepackaged and processed homograft material in dehydrated form and cut into strips of varying shapes and sizes; a porous polyethylene implant used in connection with an ocular prosthesis; a method for stimulating chondrocyte proliferation; methods where chondrocytes are grown on biodegradable fibrous polymeric matrices; and various other traditional implantation devices and methods.

In contrast to all of the above, the present invention provides a method for performing plastic surgery by obtaining a substantially three dimensional image of the target area, manipulating the substantially three dimensional image of the target area into a desired shape or profile, and making a mold of the desired shape or profile. As such, the present invention is applicable to all sorts of implant appliances and devices.

A large number of devices are described in the field of orthodontics. Tooth positioners for finishing orthodontic treatment are described by Kesling in the Am. J. Orthod. Oral. Surg. 31:297-304 (1945) and 32:285-293 (1946). The use of silicone positioners for the comprehensive orthodontic realignment of a patient's teeth is described in Warunek et al. (1989) J. Clin. Orthod. 23:694-700. Clear plastic retainers for finishing and maintaining tooth positions are commercially available from Raintree Essix, Inc., New Orleans, La. 70125, and Tru-Tain Plastics, Rochester, Minn. 55902. The manufacture of orthodontic positioners is described in U.S. Pat. Nos. 5,186,623; 5,059,118; 5,055,039; 5,035,613; 4,856,991; 4,798,534; and 4,755,139.

Other publications describing the fabrication and use of dental positioners include Kleemann and Janssen (1996) J. Clin. Orthodon. 30:673-680; Cureton (1996) J. Clin. Orthodon. 30:390-395; Chiappone (1980) J. Clin. Orthodon. 14:121-133; Shilliday (1971) Am. J. Orthodontics 59:596-599; Wells (1970) Am. J. Orthodontics 58:351-366; and Cottingham (1969) Am. J. Orthodontics 55:23-31. Kuroda et al. (1996) Am. J. Orthodontics 110:365-369 describe a method for laser scanning a plaster dental cast to produce a digital image of the cast as does U.S. Pat. No. 5,605,459.

Likewise, computer technology has been interfaced with construction of various orthodontic devices. U.S. Pat. Nos. 5,533,895; 5,474,448; 5,454,717; 5,447,432; 5,431,562; 5,395,238; 5,368,478; and 5,139,419, assigned to Ormco Corporation, describe methods for manipulating digital images of teeth for designing orthodontic appliances. U.S. Pat. No. 5,011,405 describes a method for digitally imaging a tooth and determining optimum bracket positioning for orthodontic treatment. Laser scanning of a molded tooth to produce a three-dimensional model is described in U.S. Pat. No. 5,338,198. U.S. Pat. No. 5,452,219 describes a method for laser scanning a tooth model and milling a tooth mold. Digital computer manipulation of tooth contours is described in U.S. Pat. Nos. 5,607,305 and 5,587,912. Computerized digital imaging of the jaw is described in U.S. Pat. Nos. 5,342,202 and 5,340,309. Other patents of interest include U.S. Pat. Nos. 5,549,476; 5,382,164; 5,273,429; 4,936,862; 3,860,803; 3,660,900; 5,645,421; 5,055,039; 4,798,534; 4,856,991; 5,035,613; 5,059,118; 5,186,623; and 4,755,139.

U.S. Pat. No. 6,633,789 provides a computer-implemented method that generates a computer model of one or more teeth by receiving as input a digital data set of meshes representing the teeth; selecting a curved coordinate system with mappings to and from a 3D space; and generating a function in the curved coordinate system to represent each tooth.

U.S. Pat. No. 6,802,713 provides methods and corresponding apparatus for segmenting an orthodontic treatment path into clinically appropriate substeps for repositioning the teeth of a patient. The methods include providing a digital finite element model of the shape and material of each of a sequence of appliances to be applied to a patient; providing a digital finite element model of the teeth and related mouth tissue of the patient; computing the actual effect of the appliances on the teeth by analyzing the finite elements models computationally; and evaluating the effect against clinical constraints.

### DESCRIPTION OF THE FIGURES

FIG. 1 depicts a digitally scanned face.

FIG. 2 depicts a device capable of making a three dimensional splint customized to the face of FIG. 1.

FIGS. 3A, 3B and 3C depict a three dimensional representation of a preoperative nose, a computer model of a desired nose and the resultant actual postoperative nose.

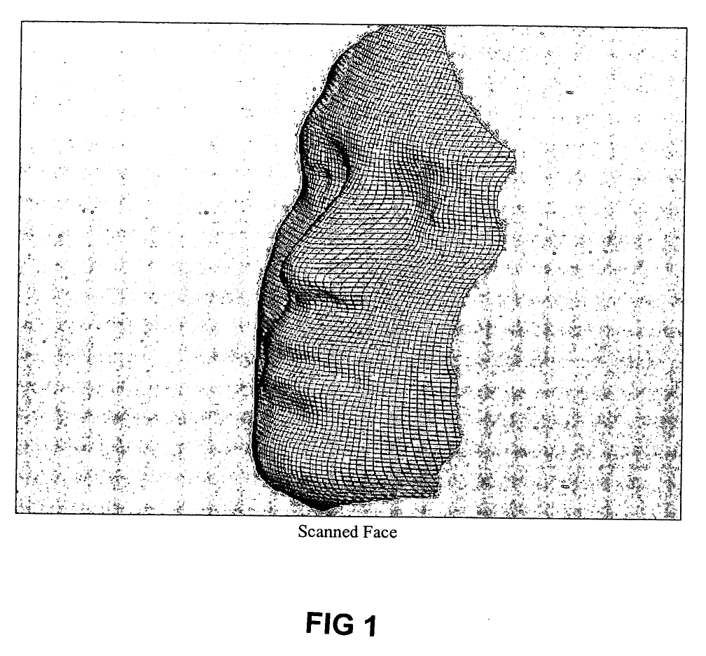

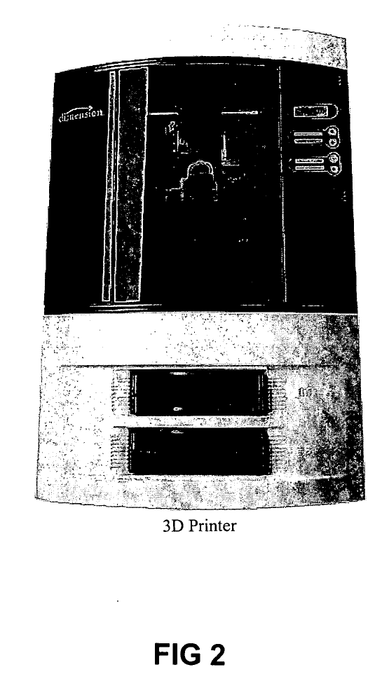

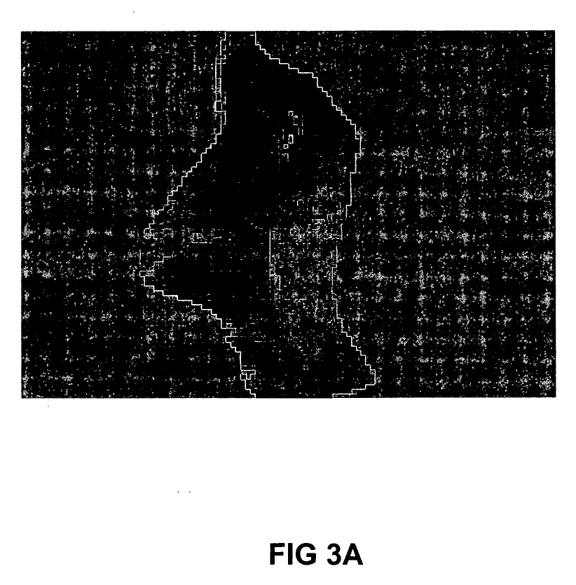

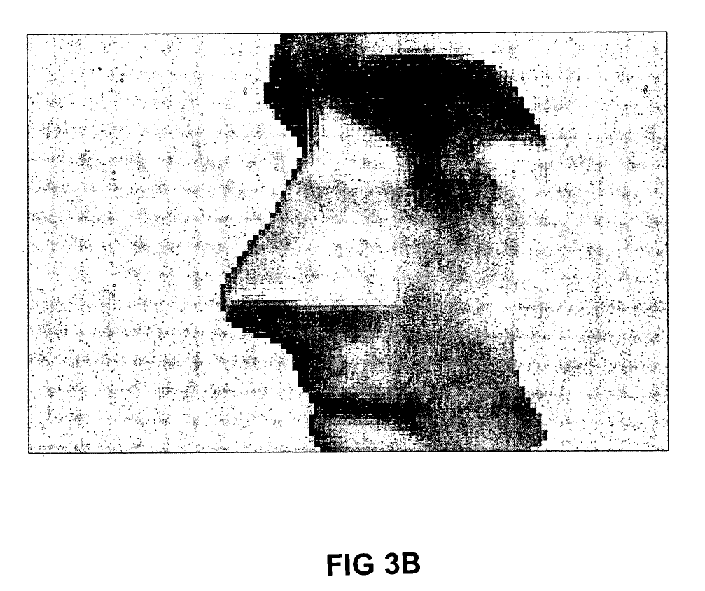

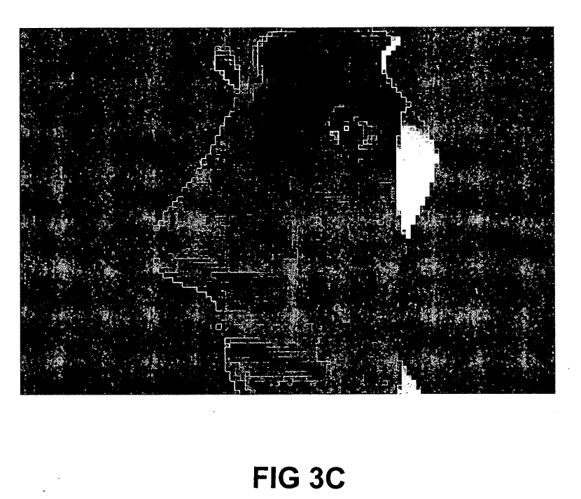

### SUMMARY OF THE INVENTION

In a first aspect, the present invention provides a method for performing plastic surgery comprising the steps of:
a) obtaining a substantially three dimensional image of the target body part or area;
b) manipulating the substantially three dimensional image of the target body part or area into a desired shape or profile; and
c) making a mold of the desired shape or profile.
   The process of obtaining a substantially three dimensional image of the target body part or area may be performed with any suitable commercially available device such as a three dimensional camera. In a preferred embodiment, the substantially three dimensional image of the target body part or area is transferred to a computer apparatus having appropriate software to allow manipulating the substantially three dimensional image of the target body part or area. Such software may be any among the various programs available at the time, however, such software is normally capable of providing a number of possible shapes, profiles and images until a desired shape or profile may be chosen. In preferred embodiments, a mold of the desired shape or profile is made using a machine capable of transferring the desired shape or profile into a model made of, for instance, plastic or rubber. The process of the instant invention is applicable to all sorts of surgeries designed to alter the shape or profile of an external body part or body surface. Particularly, the process of the instant invention is especially applicable where a negative image or model of the desired result, shape or profile is desired. For example, the process of the instant invention is especially applicable to surgical operations of the nose, ears, cheekbones, chin, and skull. Given the relative fregquency of rhinoplasty, the process of the instant invention finds frequent applications to surgeries of the nose.

In a second aspect, the present invention provides a method for performing plastic surgery comprising the steps of:
a) obtaining a substantially three dimensional image of the target body part or area;
b) manipulating the substantially three dimensional image of the target body part or area into a desired shape or profile;
c) making a mold of the desired shape or profile; and

d) using the mold of the desired shape or profile during the procedure as a guide for placing an implant into the target body part or area.

The process of the instant invention is applicable to all sorts of surgeries designed to alter the shape or profile of an external body part or body surface. Particularly, the process of the instant invention is especially applicable where a negative image or model of the desired result, shape or profile is desired. The process is especially applicable for assisting in forming a correct size and shape for an implant in the target body part or area.

In a third aspect, the present invention provides a method for performing plastic surgery comprising the steps of:
a) obtaining a substantially three dimensional image of the target body part or area;
b) manipulating the substantially three dimensional image of the target body part or area into a desired shape or profile;
c) making a mold of the desired shape or profile; and
d) using the mold of the desired shape or profile after the plastic surgery to effect or maintain the desired shape or profile of the target body part or area.
   The process of the instant invention is applicable to all sorts of surgeries designed to alter the shape or profile of an external body part or body surface. Particularly, the process of the instant invention is especially applicable where a negative image or model of the desired result, shape or profile is desired. The process is especially applicable for assisting in maintaining a size and shape for the target body part or area especially in instances where an implant is placed in the target body part or area.

In a fourth aspect, the present invention provides a mold of the desired shape or profile made according to the procedure described herein and that may be used to assist in a plastic surgery procedure. Even further, the present invention provides a mold of the desired shape or profile made according to the procedure described herein and that may be used to assist in a plastic surgery procedure or to provide a support after the plastic surgery is performed. In preferred embodiments, the mold is fabricated of plastic or rubber or some other suitable relatively hard material.

The present invention allows developing a plan for surgical treatment and communicating the same to a patient in a simple and efficient manner. It also improves clinical presentation by allowing the clinician to express treatment plans visually thereby allowing the patient to visualize changes associated with the proposed treatment. Further, it provides a tangible appliance useful in providing support for the new physiological shape produced by a surgical procedure and for maintaining the new physiological shape.

### DETAILED DESCRIPTION

The present invention provides a system and method by which a surgeon obtains a soft tissue surface three dimensional topology, alters the topology, and forms a rigid splint to use for intraoperative measurement and postoperative retention.

In particular, a paired energy emitting device and camera are used to capture the topologic morphology of a preoperative surgery patient. This image is stored as digital computer data. The data may then be transferred either to the same computer or a distant computer for processing by organic modeling CAD/CAM software

The software is used to generate paper or electronic orthogonal views of the patient for notation by the surgeon. The notation is used, either by rescanning, or by direct physical measurements, to create alterations in the topologic morphology digital model of the patient. Several models, with differing degrees of morphologic change, are then pumped to animation software to generate rotating versions of the various models. These digital motion images are recorded to transportable digital data for delivery electronically or via a DVD disk.

Once the surgeon and patient have reviewed the digital models, a selection of the desired model is made. This selected model, whose base geometry has previously been stored, is then used to generate a file for rapid prototyping.

Rapid prototyping, also known as three dimensional printing, can accept an appropriate digital data file and create, de novo, a physical example of the digitally described object. In this instance, what is asked of the prototyping hardware is to create a negative mold of the desired positive model. This accurate negative mold is then brought to the operating room.

The negative mold may be used intraoperatively as a sizer for the surgeon. The negative mold is used to size the final plastic surgical result. The negative mold may then be applied with medical grade adhesive or shaped to be held by the body itself. Optionally, the negative mold may be secured by a suture. That is, the negative mold may be sewn into the body part, especially in instances where the body part is a nose, instead of just being applied on the outside of the body part. The negative mold, now a splint, may be used both in the immediate postoperative period and for prolonged retention at night for a length of time determined by the surgeon.

### EXAMPLES

This invention may be embodied in other forms or carried out in other ways without departing from the spirit or essential characteristics thereof. The present disclosure is therefore to be considered as in all respects illustrative and not restrictive, the scope of the invention being indicated by the appended claims, and all changes which come within the meaning and range of equivalency are intended to be embraced therein.

### Example 1

### Rhinoplasty Using a Mold of the Nose Altered to Reflect the Desired Outcome

A patient sits still in a chair to have their image taken by a three dimensional camera. The picture is taken by an Inspeck (of Montreal) three dimensional camera, model “Capturor II SF.” The software driving the capture is “FAPS version 7.5” also by Inspeck. The computer driving the camera is a Windows XP computer consisting of an Athlon 2400+ processor, 1 gigabyte of RAM, and an ATI 9200 video card.

The saved files for the image are transferred to another computer for manipulation of the image obtained. This may be sent over a network. However, in this case the processing computer is in a separate location and the files are transported on a USB 256 megabyte “Cruzer Micro” portable flash drive manufactured by Sandisk. The computer uses an Inspeck program, EM 4.7, to transform the 3d image to a DXF (AutoCad) file format.

The image manipulating computer (an Athlon 3700+ processor, 1 gigabyte of RAM, and an Nvidia Quadro FX 1100 video card) uses image manipulating software (Zbrush 2 by Pixologic) to manipulate the images into four different versions of the nose. The images are given a texture and shown as if lit by a light source by software from Robert McNeal & Associates, “Flamingo 1.1.” The images are animated by software from Robert McNeal & Associates, “Bongo 1.02.” These images are based on what the surgeon feels is possible given his or her skills and the stability of the patient's tissues. The surgeon reviews a copy of the three dimensional images on paper so they can “draw” on the images their possible outcomes. Mathematical formulas of beauty such as, for example, those described in Schoenrock, Aesthetic Facial Surgery, Chapter 3 ed. Charles J. Krause; J.B. Lippincott Company, 1991, may be used to assist in formulating the desired outcome. Also, it may be feasible to “license” the shape of the noses of celebrities to use as templates.

The various images are then displayed, on an autoplaying CD with software written with Macromedia's Flash MX Professional 2004. The patient is able to see rotating versions of the nose and then select an appropriate version that may be sent via email to the surgeon.

The email is received, and software from Robert McNeal & Associates, “Rhinoceros 3.0”, is used to isolate the nose portion and create a hollow version of it, with room for nostrils. The three dimensional data is saved as an STL format file. Software from Stratasys, Catalyst, is used to transfer the STL data to the machine that builds a plastic model. The machine, a Stratasys Dimension, builds the STL image out of ABS plastic and a breakaway support plastic. This machine pulls the ABS plastic off a spool of ABS “thread”, heats it to 518 degrees Fahrenheit while rapidly moving. The ABS is used to build the hollow model. In practice, this takes about 90 minutes to perform.

The hollow model is then filled with a Platinum catalyzed Silicone based rubber, though many other final materials are possible. In some cases, Smooth-Sil 950, from Smooth-On corporation may be used. It is a 50 A durometer hardness rubber. It is cast to a thickness between 7 and 15 millimeters. It cures in 16 hours.

During the operation, the mold is used as a sizer. At the end of the operation, elastic string is strung around each of the ears and twisted twice over the dorsum of the nose to apply pressure to the splint. This is worn full time for a week and then at night for a month.

What is claimed is:

1. A method for performing surgery comprising the steps of:
a) obtaining a substantially three dimensional image of the target body part or area;
b) manipulating the substantially three dimensional image of the target body part or area into a desired shape or profile; and
c) making a mold of the desired shape or profile.

2. A method according to claim 1 wherein the manipulating of step b) is performed by transferring the substantially three dimensional image of the target body part or area to a computer apparatus having appropriate software to allow manipulating the substantially three dimensional image of the target body part or area.

3. A method according to claim 1 wherein the making of step c) is performed by making a mold of the desired shape or profile using a machine capable of transferring the desired shape or profile into a model made of a plastic or a rubber.

4. A method according to claim 1 wherein the target body part or area is selected from the group consisting of a nose, ear, cheekbone, chin, and skull.

5. A method for performing plastic surgery comprising the steps of:
a) obtaining a substantially three dimensional image of the target body part or area;
b) manipulating the substantially three dimensional image of the target body part or area into a desired shape or profile;
c) making a mold of the desired shape or profile; and
d) using the mold of the desired shape or profile during the procedure as a guide for placing an implant into the target body part or area.

6. A method according to claim 5 wherein the manipulating of step b) is performed by transferring the substantially three dimensional image of the target body part or area to a computer apparatus having appropriate software to allow manipulating the substantially three dimensional image of the target body part or area.

7. A method according to claim 5 wherein the making of step c) is performed by making a mold of the desired shape or profile using a machine capable of transferring the desired shape or profile into a model made of a plastic or a rubber.

8. A method according to claim 5 wherein the target body part or area is selected from the group consisting of a nose, ear, cheekbone, chin, and skull.

9. A method for performing plastic surgery comprising the steps of:
a) obtaining a substantially three dimensional image of the target body part or area;
b) manipulating the substantially three dimensional image of the target body part or area into a desired shape or profile;
c) making a mold of the desired shape or profile; and
d) using the mold of the desired shape or profile after the plastic surgery to effect or maintain the desired shape or profile of the target body part or area.

10. A method according to claim 9 wherein the manipulating of step b) is performed by transferring the substantially three dimensional image of the target body part or area to a computer apparatus having appropriate software to allow manipulating the substantially three dimensional image of the target body part or area.

11. A method according to claim 9 wherein the making of step c) is performed by making a mold of the desired shape or profile using a machine capable of transferring the desired shape or profile into a model made of a plastic or a rubber.

12. A method according to claim 9 wherein the target body part or area is selected from the group consisting of a nose, ears, cheekbones, chin, and skull.

13. A method according to claim 9 wherein the target body part or area is selected from the group consisting of a nose, ears, cheekbones, chin, and skull.

14. A mold of the desired shape or profile made according to the procedure of claim 1.

15. A mold according to claim 14 wherein the target body part or area is selected from the group consisting of a nose, ears, cheekbones, chin, and skull.

16. A mold according to claim 14 wherein the target body part or area is a nose.
